(19) United States
(12) Patent Application Publication
(10) Pub. No.: US 2023/0045372 A1
(43) Pub. Date: Feb. 9, 2023

(54) METHOD AND DEVICE FOR PROVIDING CUSTOMIZED SOLUTION FOR IMPROVING INTESTINAL ENVIRONMENT

(71) Applicant: HEM PHARMA INC., Pohang-si, Gyeongsangbuk-do (KR)

(72) Inventors: So Young PARK, Suwon-si, Gyeonggi-do (KR); Yo Sep JI, Suwon-si, Gyeonggi-do (KR)

(21) Appl. No.: 17/793,836

(22) PCT Filed: Dec. 15, 2020
(86) PCT No.: PCT/KR2020/018343

§ 371 Date: Jul. 19, 2022

(30) Foreign Application Priority Data
Jan. 23, 2020 (KR) 10-2020-0009519

(51) Int. Cl. G16H 50/30; G16H 10/60; G16H 50/20; G16H 20/60

(52) U.S. Cl. CPC G16H 50/30; G16H 10/60; G16H 50/20; G16H 20/60

(57) ABSTRACT

A method for providing a personalized solution for improving an intestinal environment based on a postbiotic-based health index includes receiving registration of a kit for collecting a sample from a user device; calculating a postbiotic-based health index of at least one culture in which the sample is treated with at least one intestinal environment-improving candidate material; and recommending at least one product for improving an intestinal environment, including the at least one intestinal environment-improving candidate material based on the postbiotic-based health index of the at least one culture.

### TECHNICAL FIELD

The present disclosure relates to a method and server for providing a personalized solution for improving an intestinal environment.

### BACKGROUND

Microbiota is known to play an important role in maintaining the homeostasis of the host (human) immunity, metabolites and the like. The microbiota and the host transmit and receive chemical signals to and from each other, and the expression of immune cells, production of neurotransmitter and production of short chain fatty acids (SCFA) by the microbiota have a significant effect on the host system.

Probiotics/prebiotics balance the host's unbalanced microbiota so that a healthy metabolite of the microbiota boosts the host's health.

Existing probiotics, like generic drugs, give everyone the same dose and similar species.

However, per-human microbiome similarity is less than 50%, and each person's intestinal environment is different. Accordingly, it is difficult for people who have taken the same probiotics to get the same effect. Therefore, recently, there is increasing recognition and research on the personalization of probiotics.

(Patent Document 1) Korean Patent Laid-open Publication No. 2019-0004586 (published on Jan. 14, 2019)

### DISCLOSURE OF THE INVENTION

### Problems to be Solved by the Invention

The present disclosure is to solve the problems of the prior art described above, and to provide a personalized solution for improving an intestinal environment based on a postbiotic-based health index of a culture in which a sample is treated with at least one intestinal environment-improving candidate material. The problems to be solved by the present disclosure are not limited to the above-described problems. There may be other problems to be solved by the present disclosure.

### Means for Solving the Problems

As a means for solving the problems, according to an aspect of the present disclosure, a method for providing a personalized solution for improving an intestinal environment based on a postbiotic-based health index includes receiving registration of a kit for collecting a sample from a user device; calculating a postbiotic-based health index of at least one culture in which the sample is treated with at least one intestinal environment-improving candidate material; and recommending at least one product for improving an intestinal environment, including the at least one intestinal environment-improving candidate material based on the postbiotic-based health index of the at least one culture.

According to another aspect of the present disclosure, a solution providing server for providing a personalized solution for improving an intestinal environment based on a postbiotic-based health index includes a kit registration unit that receives registration of a kit for collecting a sample from a user device; a postbiotic-based health index calculation unit that calculates a postbiotic-based health index of at least one culture in which the sample is treated with at least one intestinal environment-improving candidate material; and a product recommendation unit that recommends at least one product for improving an intestinal environment, including the at least one intestinal environment-improving candidate material based on the postbiotic-based health index of the at least one culture.

The above-described aspects are provided by way of illustration only and should not be construed as liming the present disclosure. Besides the above-described embodiments, there may be additional embodiments described in the accompanying drawings and the detailed description.

### Effects of the Invention

According to any one of the above-described means for solving the problems of the present disclosure, the present disclosure provides a personalized solution for improving an intestinal environment based on a postbiotic-based health index of a culture in which a sample is treated with at least one intestinal environment-improving candidate material.

Accordingly, the present disclosure makes it possible to check what the intestinal environment-improving candidate material required for a user's intestine is according to the postbiotic-based health index of the culture treated with the intestinal environment-improving candidate material. Therefore, it is possible to recommend a personalized product for improving an intestinal environment.

### BRIEF DESCRIPTION OF THE DRAWINGS

FIG. 1 is a configuration diagram of a system for providing a personalized solution for improving an intestinal environment according to an embodiment of the present disclosure.

FIG. 2 is a block diagram of a solution providing server shown in FIG. 1 according to an embodiment of the present disclosure.

FIG. 3 is a diagram for explaining a method for providing a personalized solution for improving an intestinal environment according to an embodiment of the present disclosure.

FIG. 4A is a diagram for explaining a method for selecting an intestinal environment-improving candidate material for improving the intestine according to an embodiment of the present disclosure.

FIG. 4B is a diagram for explaining a method for selecting an intestinal environment-improving candidate material for improving the intestine according to an embodiment of the present disclosure.

FIG. 4C is a diagram for explaining a method for selecting an intestinal environment-improving candidate material for improving the intestine according to an embodiment of the present disclosure.

FIG. 4D is a diagram for explaining a method for selecting an intestinal environment-improving candidate material for improving the intestine according to an embodiment of the present disclosure.

FIG. 5A is a diagram for explaining a method for providing intestinal environment result information according to an embodiment of the present disclosure.

FIG. 5B is a diagram for explaining a method for providing intestinal environment result information according to an embodiment of the present disclosure.

FIG. 5C is a diagram for explaining a method for providing intestinal environment result information according to an embodiment of the present disclosure.

FIG. 5D is a diagram for explaining a method for providing intestinal environment result information according to an embodiment of the present disclosure.

FIG. 6 is a diagram for explaining a method for recommending a product for improving an intestinal environment and providing an expected effect of the recommended product according to an embodiment of the present disclosure.

FIG. 7 is a flowchart showing a method for providing a personalized solution for improving an intestinal environment according to an embodiment of the present disclosure.

### BEST MODE FOR CARRYING OUT THE INVENTION

Hereafter, embodiments of the present disclosure will be described in detail with reference to the accompanying drawings so that the present disclosure may be readily implemented by a person with ordinary skill in the art. However, it is to be noted that the present disclosure is not limited to the embodiments but may be embodied in various other ways. In drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “unit” includes a unit implemented by hardware, a unit implemented by software, and a unit implemented by both of them. One unit may be implemented by two or more pieces of hardware, and two or more units may be implemented by one piece of hardware.

Through the whole document, a part of an operation or function described as being carried out by a terminal or device may be carried out by a server connected to the terminal or device. Likewise, a part of an operation or function described as being carried out by a server may be carried out by a terminal or device connected to the server.

Hereinafter, the present disclosure will be explained in detail with reference to the accompanying configuration views or process flowcharts.

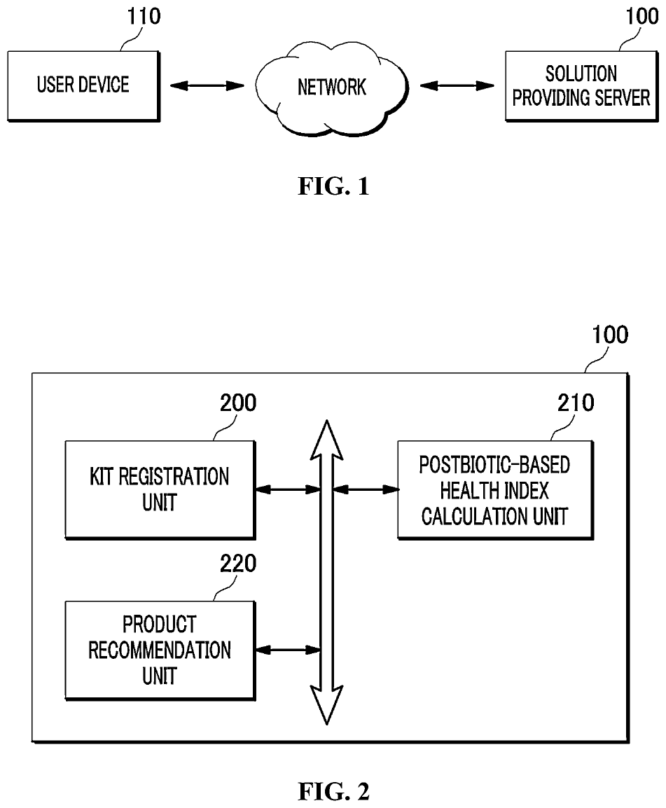

FIG. 1 is a configuration diagram of a system for providing a personalized solution for improving an intestinal environment according to an embodiment of the present disclosure.

Referring to FIG. 1, a personalized solution providing system may include a solution providing server 100 and a user device 110. However, the personalized solution providing system illustrated in FIG. 1 is just an embodiment of the present disclosure and should not be construed as liming the present disclosure and can be configured differently from that shown in FIG. 1 according to various embodiments of the present disclosure.

The components of the personalized solution providing system illustrated in FIG. 1 are typically connected to each other via a network (not shown). The network refers to a connection structure that enables information exchange between nodes such as devices and servers, and includes LAN (Local Area Network), WAN (Wide Area Network), Internet (WWW: World Wide Web), a wired or wireless data communication network, a telecommunication network, a wired or wireless television network, and the like. Examples of the wireless data communication network may include 3G, 4G, 5G, 3GPP (3rd Generation Partnership Project), LTE (Long Term Evolution), WIMAX (World Interoperability for Microwave Access), Wi-Fi, Bluetooth communication, infrared communication, ultrasonic communication, VLC (Visible Light Communication), LiFi, and the like, but may not be limited thereto.

The user device 110 may access a personalized intestinal solution providing application or personalized solution providing web page in order to receive a personalized intestinal solution service, receive information about a kit to be used for examining a user's intestinal environment from the user, and transmit the received information about the kit to the solution providing server 100. Herein, the kit is a tool used to collect the user's sample. Herein, the sample is human feces and composed of materials present in the intestine, such as microbiota and intestinal microbial metabolites (short chain fatty acids (SCFA)).

When the user device 110 registers the kit for collecting the user's sample in the solution providing server 100, the solution providing server 100 may calculate a postbiotic-based health index of at least one culture in which the sample is treated with at least one intestinal environment-improving candidate material. Herein, the at least one intestinal environment-improving candidate material may be a material in which at least one of probiotics or prebiotics, such as Lactobacillus and Bifidobacterium, is mixed at different concentration and amount ratios.

The solution providing server 100 may recommend at least one product for improving an intestinal environment, which includes at least one intestinal environment-improving candidate material effective in improving the user's intestinal environment, to the user device 110 based on the postbiotic-based health index of at least one culture.

Hereinafter, the operation of each component of the personalized solution providing system of FIG. 1 will be described in more detail.

FIG. 2 is a block diagram of the solution providing server 100 shown in FIG. 1 according to an embodiment of the present disclosure.

Referring to FIG. 2, the solution providing server 100 may include a kit registration unit 200, a postbiotic-based health index calculation unit 210 and a product recommendation unit 220. However, the solution providing server 100 depicted in FIG. 2 is just one of embodiments of the present disclosure and can be modified in various ways based on the components depicted in FIG. 2.

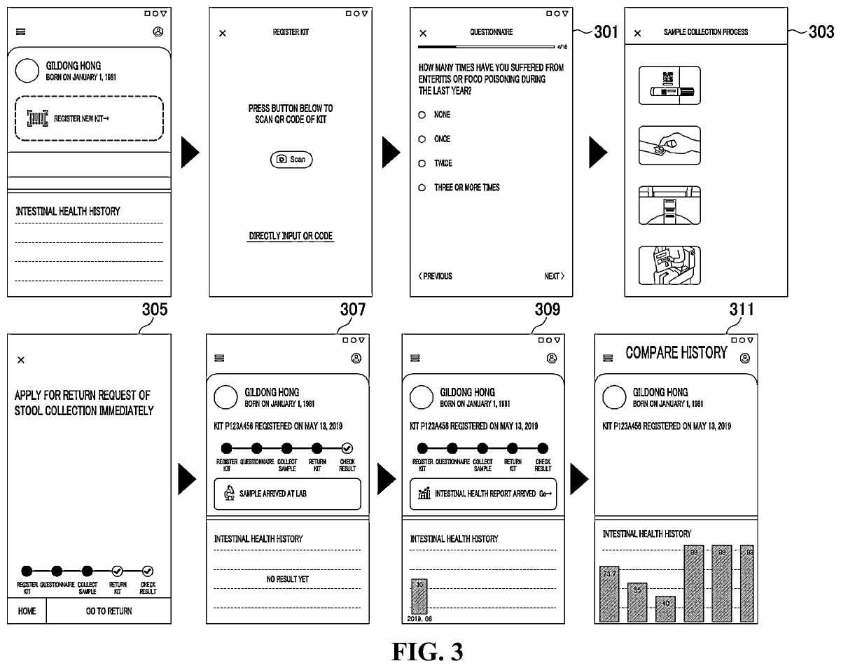

Hereinafter, FIG. 2 will be described with reference to FIG. 3 through FIG. 6.

The kit registration unit 200 may receive registration of a kit for collecting a sample from the user device 110. Herein, the sample is human feces and composed of materials present in the intestine, such as microbiota, intestinal microbial metabolites and short chain fatty acids.

For example, referring to FIG. 2, the kit registration unit 200 may receive registration of information about a kit for examining the user's intestinal environment from the user device 110 which has accessed the personalized intestinal solution providing application or personalized solution providing web page. In this case, the user device 110 may log into the personalized intestinal solution providing application or personalized solution providing web page in order to receive the personalized intestinal solution service, and may register information about the kit to be used by the user to examine the intestinal environment via the personalized intestinal solution providing application or personalized solution providing web page.

The kit registration unit 200 may receive, from the user device 110, a digital code (e.g., QR code, etc.) displayed on the kit scanned by a camera of the user device 110. Herein, the digital code may include information about the kit (e.g., identification information of the kit, etc.).

A medical examination information receiving unit (not shown) may receive medical examination information 301 of the user from the user device 110. Here, the medical examination information 301 may include a plurality of questions for checking the user's current intestinal health conditions and the user's answers to the respective questions.

A guide unit (not shown) may guide the user device 110 through a method 303 for collecting a sample using the kit via the personalized intestinal solution providing application or personalized solution providing web page.

After the user's sample is collected using the kit, the guide unit (not shown) may guide the user device 110 through a method 305 for requesting a return of the kit with the collected sample to a laboratory (i.e., laboratory analyzing intestinal microbiota). That is, the user may put the kit with the sample into a delivery bag according to the method and apply for a return request via the personalized intestinal solution providing application or personalized solution providing web page. Then, the user's kit may be returned to the laboratory through a courier company.

The guide unit (not shown) may guide the user device 110 through return process information 307 of the kit with the collected sample of the user and analysis result information 309 of the user's sample via the personalized intestinal solution providing application or personalized solution providing web page.

When the user undergoes an intestinal environment examination several times, the guide unit (not shown) may provide the user device 110 with history information 311 so that the sample analysis results for the respective examinations can be compared with each other.

Referring to FIG. 2 again, the postbiotic-based health index calculation unit 210 may use Personalized Pharmaceutical Meta-Analysis Screening (PMAS) to calculate a postbiotic-based health index of at least one culture in which a sample is treated with at least one intestinal environment-improving candidate material.

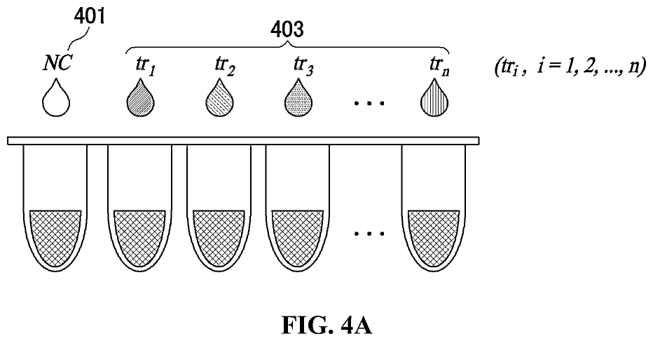

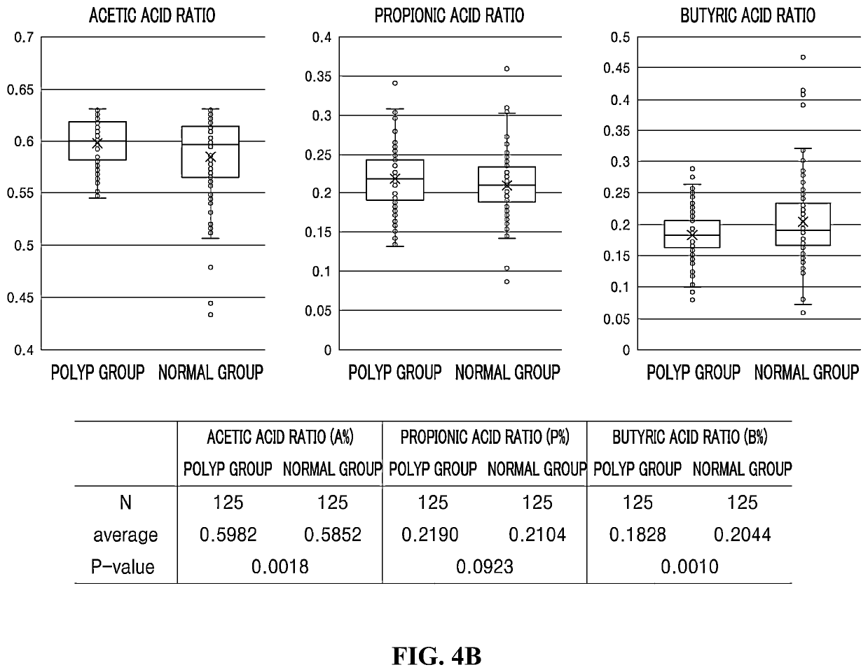

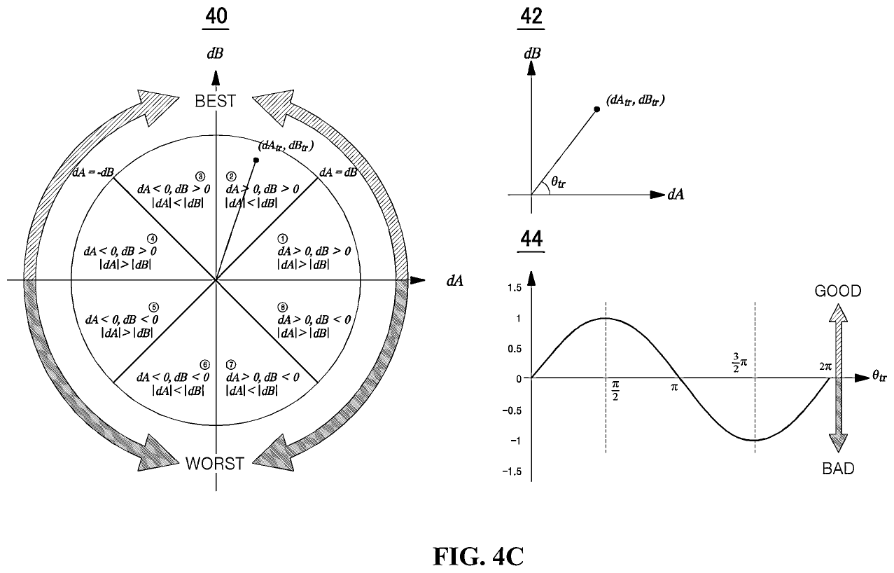

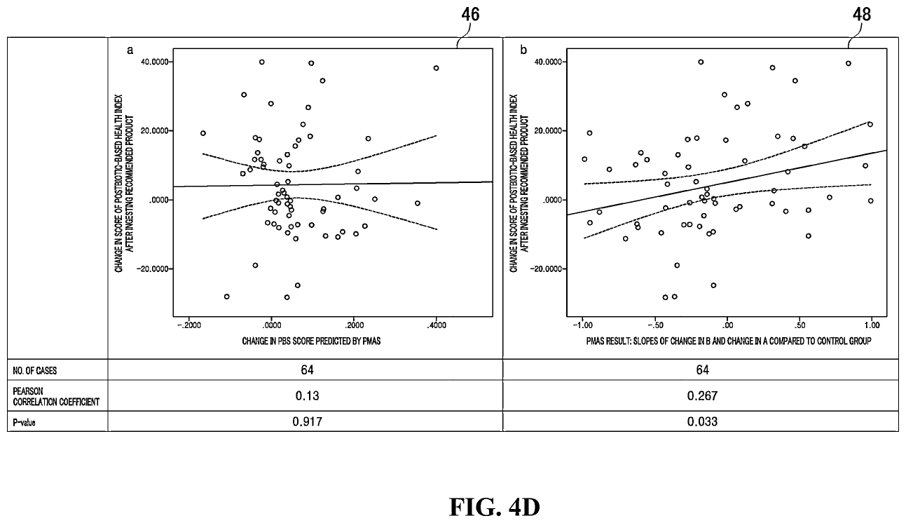

Referring to FIG. 4A, when a sample is mixed with a composition for screening an intestinal environment-improving material, the mixture is treated with each intestinal environment-improving candidate material 403 and then cultured. Herein, the composition is prepared for screening a candidate material capable of improving an intestinal environment, and it may be a composition to be used in a series of processes of monitoring the progress of improvement in the intestinal environment and evaluating whether the candidate material can improve the intestinal environment. Also, the composition is prepared for identically or similarly mimicking the intestinal environment of an individual user in vitro and makes is possible to accurately and efficiently check whether the candidate material can improve the intestinal environment under in vitro conditions. Herein, each of the intestinal environment-improving candidate materials 403 may be a material in which at least one of probiotics or prebiotics, such as Lactobacillus and Bifidobacterium, is mixed at different concentration and amount ratios.

Further, a control group 401 is prepared for comparison with the culture in which the sample is treated with the intestinal environment-improving candidate material 403. Herein, the control group 401 may be a sample (i.e., sample without any treatment) that can be compared in changes of the intestinal environment caused by treatment with the intestinal environment-improving candidate material 403.

### First Example

According to First Example, the postbiotic-based health index calculation unit 210 may calculate the postbiotic-based health index of a culture of the control group 401 and calculate the postbiotic-based health index of each culture cultured with each intestinal environment-improving candidate material 403. In this case, the postbiotic-based health index may be an index based on the ratio of short chain fatty acids (SCFA) in the culture. Herein, the SCFA refers to a short-length fatty acid with six or fewer carbon atoms and is a representative metabolite produced from intestinal microbiota. Also, the SCFA has useful functions in the body, such as an increase in immunity, stabilization of intestinal lymphocytes, a decrease in insulin signaling, and stimulation of sympathetic nerves. Representative examples of the SCFA may include acetic acid (acetate), propionic acid (propionate) and butyric acid (butyrate), but the present disclosure is not limited thereto. Herein, the postbiotic-based health index may be an index based on the ratio of acetic acid (acetate) to propionic acid (propionate) and butyric acid (butyrate).

The postbiotic-based health index calculation unit 210 may measure the SCFA constituting the culture of the control group 401 and measure the SCFA constituting each culture treated and cultured with each intestinal environment-improving candidate material 403. Also, the postbiotic-based health index calculation unit 210 may calculate the contents of propionic acid (propionate), butyric acid (butyrate) and acetic acid (acetate), respectively, in the SCFA of the culture of the control group 401 and calculate the contents of propionic acid (propionate), butyric acid (butyrate) and acetic acid (acetate), respectively, in the SCFA of each culture treated and cultured with each intestinal environment-improving candidate material 403.

The postbiotic-based health index calculation unit 210 may calculate the ratio of acetic acid (acetate) to propionic acid (propionate) and butyric acid (butyrate) constituting the SCFA in the culture of the control group 401 and may calculate the ratio of acetic acid (acetate) to propionic acid (propionate) and butyric acid (butyrate) constituting the SCFA in each culture cultured with each intestinal environment-improving candidate material 403. Herein, the ratio of acetic acid (acetate) Aᵣₐₜᵢₒ can be calculated according to Equation 1.

$${A}_{ratio} = \frac{Acetic acid ( mM )}{\begin{matrix}Acetic acid ( mM ) + \\ Propionic acid ( mM ) + Butyric acid ( mM )\end{matrix}} \qquad [ Equation 1 ]$$

Herein, acetic acid is the content of acetic acid in the SCFA of the culture, propionic acid is the content of propionic acid in the SCFA of the culture and butyric acid is the content of butyric acid in the SCFA of the culture, and Aᵣₐₜᵢₒ is a ratio value of acetic acid to propionic acid and butyric acid.

The postbiotic-based health index calculation unit 210 may calculate a postbiotic-based health index of the culture of the control group 401 based on the ratio value of acetic acid in the culture of the control group 401 and calculate a postbiotic-based health index of each culture cultured with each intestinal environment-improving candidate material 403 based on the ratio value of acetic acid in each culture cultured with each intestinal environment-improving candidate material 403. Herein, when the ratio value of acetic acid is substituted into Equation 2, a postbiotic-based health index is calculated.

$$PBS = \frac{100}{0.6} ⋆ ( 0 \text{.6} - \vert {A}_{ratio} - 0.5 \; 85 \vert ) \qquad [ Equation 2 ]$$

Herein, Aᵣₐₜᵢₒ is a ratio value of acetic acid in die culture, and the postbiotic-based health index is the postbiotic-based health index of the culture.

Referring to Equation 2, the postbiotic-based health index has the highest value when the ratio of acetic acid is 58.5%, and the values decreases as the ratio of acetic acid goes away from 58.5%.

In this regard, the present applicant followed up the ratio of acetic acid, propionic acid and butyric acid in a polyp group in which polyps were found in the colon and in a normal group without polyps after colonoscopy. Referring to FIG. 4B, the present applicant compared the average of the polyp group with the average of the normal group through the follow-up examination and found a significant difference (p-value=0.0018) in acetic acid ratio from among the acetic acid ratio, the propionic acid ratio and the butyric acid ratio. As a result, the present applicant determined that when the average value of the acetic acid ratio in the normal group is 0.585, the postbiotic-based health index is at an appropriate level.

The postbiotic-based health index calculation unit 210 may determine information about changes in SCFA ratio based on the calculated postbiotic-based health index of each culture to determine whether each intestinal environment-improving candidate material 403 can improve microbiota. Herein, the microbiota plays an important role in maintaining the homeostasis, e.g., human immunity, metabolites and the like. The microbiota and the host transmit and receive chemical signals to and from each other, and the expression of immune cells, neurotransmitter production and short chain fatty acids (SCFA) by the microbiota have a significant effect on the host system.

The postbiotic-based health index calculation unit 210 may calculate the difference between the postbiotic-based health index of each culture treated with each intestinal environment-improving candidate material 403 and the postbiotic-based health index of the culture of the control group 401 as in Equation 3.

dPBSₜᵣᵢ=PBSₜᵣᵢ−PBS_{NC}, i=1,2, . . . ,n [Equation 3]

Herein, PBSₜᵣᵢ is the postbiotic-based health index of the culture treated with an ith intestinal environment-improving candidate material 403 and PBS_{NC} is the postbiotic-based health index of the culture of the control group 401.

The postbiotic-based health index calculation unit 210 may select at least one culture based on the difference between the postbiotic-based health index of the control group 401 without any treatment with an intestinal environment-improving candidate material and the postbiotic-based health index of at least one culture treated with the intestinal environment-improving candidate material 403. For example, the postbiotic-based health index calculation unit 210 may select a culture in which the sample is treated with the intestinal environment-improving candidate material 403 and which has the greatest difference between a postbiotic-based health index of the control group 401 in which the sample is not treated with any intestinal environment-improving candidate material and the postbiotic-based health index of the at least one culture.

The product recommendation unit 220 may recommend at least one product for improving an intestinal environment, including at least one intestinal environment-improving candidate material based on the postbiotic-based health index of the selected at least one culture. Herein, the product for improving an intestinal environment may include at least one of probiotics, prebiotics, food, health functional food and drug.

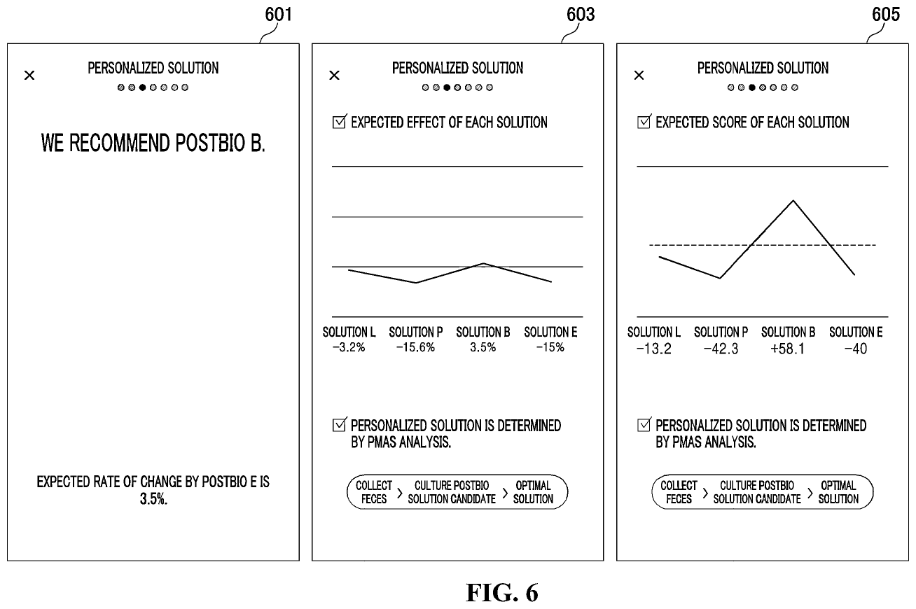

For example, referring to FIG. 6, the product recommendation unit 220 may provide the user device 110 with information about a recommended product (e.g., postbio B product) including an intestinal environment-improving candidate material effective in improving the user's intestinal environment and information 601 about an expected rate of change in improvement of the intestinal environment when the recommended product is taken.

The product recommendation unit 220 may recommend a product for improving an intestinal environment including an intestinal environment-improving candidate material used for culturing a culture having the greatest difference between a postbiotic-based health index of the control group 401 and the postbiotic-based health index of the at least one culture.

For example, the product recommendation unit 220 may provide the user device 110 with information 603 about the effect when the product recommendation unit 220 recommends the product including the intestinal environment-improving candidate material used for culturing the culture having the greatest difference between a postbiotic-based health index of the control group 401 and the postbiotic-based health index of the at least one culture.

### Second Example

According to Second Example, the postbiotic-based health index calculation unit 210 may calculate the postbiotic-based health index based on the increase or decrease and the content of each of acetic acid and butyric acid in at least one culture in which the sample is treated with at least one intestinal environment-improving candidate material.

Referring to FIG. 4C, the postbiotic-based health index calculation unit 210 may calculate difference values between the contents of acetic acid Aₜᵣᵢ and butyric acid Bₜᵣᵢ of the sample treated with each intestinal environment-improving candidate material and the contents of acetic acid A_{NC} and butyric acid B_{NC}, respectively, as in Equation 4.

dAₜᵣᵢ=Aₜᵣᵢ−A_{NC}, i=1,2, . . . ,n

dBₜᵣᵢ=Bₜᵣᵢ−B_{NC}, i=1,2, . . . ,n [Equation 4]

When a coordinate point (dAₜᵣᵢ, dBₜᵣᵢ) of the difference value in acetic acid and the difference value in butyric acid for each intestinal environment-improving candidate material is displayed on a coordinate plane, the postbiotic-based health index calculation unit 210 may determine what effect each intestinal environment-improving candidate material has depending on the position of the corresponding coordinate point.

As for the intestinal SCFA ratio, it is known that people with colorectal cancer or polyps have a higher acetic acid ratio and a lower butyric acid ratio than healthy people. Therefore, in the present disclosure, not only the increase or decrease of acetic acid or butyric acid, but also the amount of change of these two values is considered. For example, reference numeral 40 in FIG. 4C is a diagram illustrating a coordinate plane in which a sample treated with an intestinal environment-improving candidate material and a sample of a control group are divided into eight sections.

Sections \textcircled{1} to \textcircled{4} are cases where the amount of butyric acid in the sample treated with the intestinal environment-improving candidate material increases. Section \textcircled{1} corresponds to a case where both the amount of acetic acid and the amount of butyric acid increase, and an increase in acetic acid |dA| is greater than an increase in butyric acid |dB|. Section \textcircled{2} corresponds to a case where both the amount of acetic acid and the amount of butyric acid increase, and the increase in butyric acid is greater than the increase in acetic acid. Section \textcircled{3} corresponds to a case where the amount of acetic acid decreases and the amount of butyric acid increases, and the increase in butyric acid is greater than a decrease in acetic acid. Section \textcircled{4} corresponds to a case where the amount of acetic acid decreases and the amount of butyric acid increases, and the decrease in acetic acid is greater than the increase in butyric acid.

In the case of \textcircled{1} to \textcircled{4}, the postbiotic-based health index calculation unit 210 may determine that when the increase in butyric acid is greater than the increase (or decrease) in acetic acid, it means a good situation. Also, the postbiotic-based health index calculation unit 210 may determine that being closer to the dB axis means the intestinal environment-improving candidate material is more effective.

Sections \textcircled{5} to \textcircled{8} are cases where the amount of butyric acid in the sample treated with the intestinal environment-improving candidate material decreases. Section \textcircled{5} corresponds to a case where both the amount of acetic acid and the amount of butyric acid decrease, and the decrease in acetic acid is greater than a decrease in butyric acid. Section \textcircled{6} corresponds to a case where both the amount of acetic acid and the amount of butyric acid decrease, and the decrease in butyric acid is greater than the decrease in acetic acid. Section \textcircled{7} corresponds to a case where the amount of acetic acid increases and the amount of butyric acid decreases, and the increase in butyric acid is greater than the decrease in acetic acid. Section \textcircled{8} corresponds to a case where the amount of acetic acid increases and the amount of butyric acid decreases, and the increase in acetic acid is greater than the decrease in butyric acid.

In the case of \textcircled{5} to \textcircled{8}, the postbiotic-based health index calculation unit 210 may determine that when the decrease in butyric acid is smaller than the increase (or decrease) in acetic acid, it means a worse situation. Also, the postbiotic-based health index calculation unit 210 may determine that being closer to the dB axis means the intestinal environment-improving candidate material is less effective.

For example, it may be determined that an intestinal environment-improving candidate material with a difference value in butyric acid being greater than 0 and close to the Best axis (dB axis) is effective, and an intestinal environment-improving candidate material with a difference value in butyric acid being smaller than 0 and close to the Worst axis (dB axis) is not effective.

Referring to reference numerals 42 and 44 of FIG. 4C, when an angle between the coordinate point (dAₜᵣᵢ, dBₜᵣᵢ) for each intestinal environment-improving candidate material and an X-axis is θₜᵣᵢ, the postbiotic-based health index calculation unit 210 may calculate θₜᵣᵢ according to Equation 5. The value of butyric acid is usually ⅓ lower than that of acetic acid, and, thus, θₜᵣᵢ can be calculated by multiplying dBₜᵣᵢ by three.

$${θ}_{tri} = {tan}^{- 1} \frac{d {B}_{tri} \times 3}{{dA}_{tri}} , i = 1 , 2 , … , n \qquad [ Equation 5 ]$$

The postbiotic-based health index calculation unit 210 may calculate a value of sin θₜᵣᵢ based on θₜᵣᵢ calculated according to Equation 5. The postbiotic-based health index calculation unit 210 may determine that as the value of sin θₜᵣᵢ is closer to 1, the intestinal environment-improving candidate material is more effective, and as the value of sin θₜᵣᵢ is closer to −1, the intestinal environment-improving candidate material is less effective.

The postbiotic-based health index calculation unit 210 may calculate a value of sin θₜᵣᵢ for each culture treated with each intestinal environment-improving candidate material and may select an intestinal environment-improving candidate material having the highest sin θₜᵣᵢ.

The product recommendation unit 220 may recommend at least one product for improving an intestinal environment, including the intestinal environment-improving candidate material having the highest sin θₜᵣᵢ. Herein, the product for improving an intestinal environment may include at least one of probiotics, prebiotics, food, health functional food and drug.

For example, referring to FIG. 6, the product recommendation unit 220 may provide the user device 110 with information 605 about the effect when the product recommendation unit 220 recommends the product including the intestinal environment-improving candidate material having the highest sin θₜᵣᵢ.

The present applicant verified the effect of the present disclosure by conducting a mini clinical trial on 64 people. FIG. 4D shows the results of this mini clinical trial.

Referring to FIG. 4D, reference numeral 46 denotes a change in score of the postbiotic-based health index after ingesting the recommended product according to First Example, and reference numeral 48 denotes a change in score of the postbiotic-based health index after ingesting the recommended product according to Second Example.

The postbiotic-based health index calculation unit 210 may calculate the ratio of SCFA included in the user's sample and calculate the ratio of acetic acid in the calculated ratio of SCFA.

The postbiotic-based health index calculation unit 210 may calculate the ratio of acetic acid to propionic acid and butyric acid in the SCFA included in the user's sample according to Equation 1 to Equation 2 and calculate the user's postbiotic-based health index based on the calculated ratio of acetic acid.

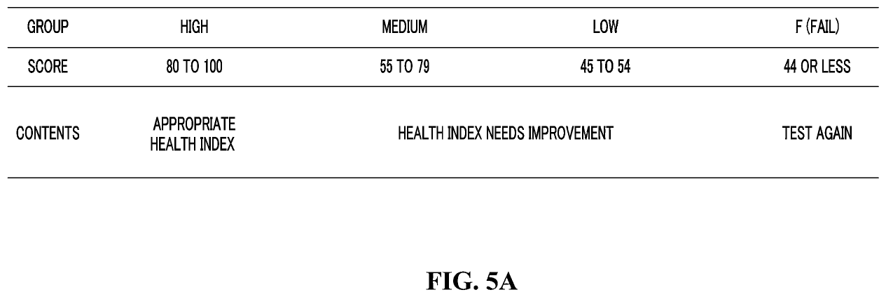

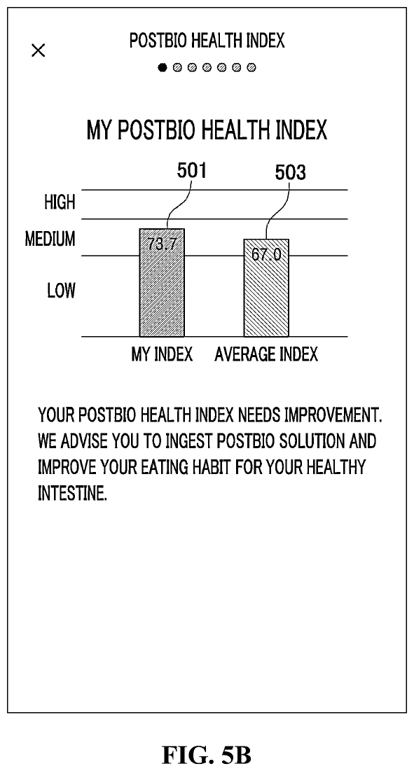

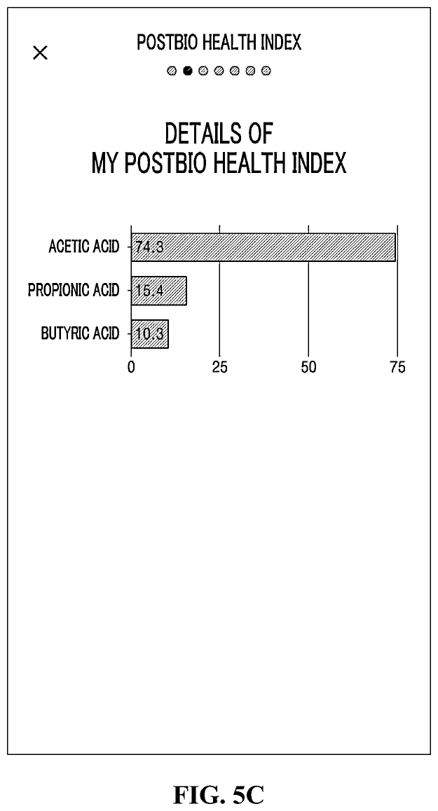

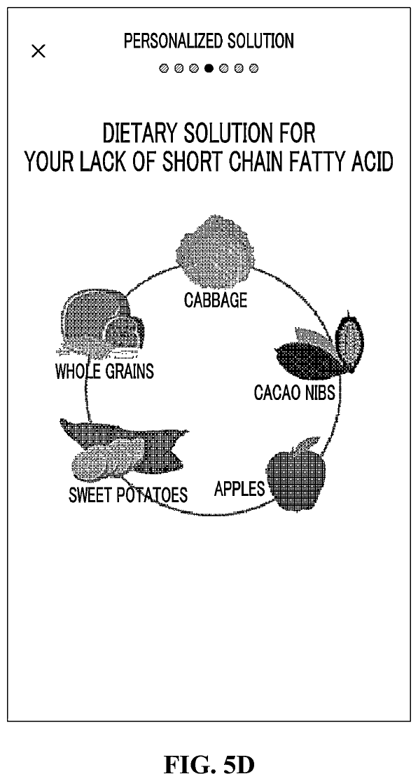

Referring to FIG. 5A and FIG. 5B, the postbiotic-based health index calculation unit 210 may classify the postbiotic-based health index of the user into any one of a plurality of predetermined groups. An intestinal result providing unit (not shown) may compare information 501 about the user's postbiotic-based health index with information 503 about the average postbiotic-based health index based on the identified group and provide the user device 110 with the result of comparison.

Referring to FIG. 5C, the intestinal result providing unit (not shown) may provide the user device 110 with information about the ratio of SCFA of the user, which is a factor in calculating the user's postbiotic-based health index. Further, the intestinal result providing unit (not shown) may provide the user device 110 with index information of each of the ratios of acetic acid, propionic acid and butyric acid constituting the SCFA of the user.

Referring to FIG. 5A and FIG. 5D, the intestinal result providing unit (not shown) may provide the user's dietary solution information depending on a group to which the user's postbiotic-based health index belongs among the plurality of predetermined groups. For example, referring to FIG. 5A, if the user's postbiotic-based health index belongs to a “medium or low” group, it means short chain fatty acids are insufficient in the user's intestine, and, thus, the intestinal result providing unit (not shown) may provide the user device 110 with information about food supplementing fatty acids (e.g., a solution recommending ingestion of cabbage, whole grains, sweet potatoes, apples, cacao nibs, etc.).

Alternatively, if the user's postbiotic-based health index belongs to a “high” group, the intestinal result providing unit (not shown) may provide a Lactobacillus solution for the low level of Lactobacillus or Bifidobacterium in the user's intestine (e.g., a solution recommending ingestion of cheese, kefir, kimchi, yogurt, etc. if Lactobacillus is at a low level), and if Lactobacillus and Bifidobacterium are at the same level, the intestinal result providing unit (not shown) may provide a Bifidobacterium solution (e.g., a solution recommending ingestion of berries, chicory, onions, carrots, garlic, etc.).

Meanwhile, it would be understood by a person with ordinary skill in the art that each of the kit registration unit 200, the postbiotic-based health index calculation unit 210 and the product recommendation unit 220 can be implemented separately or in combination with one another.

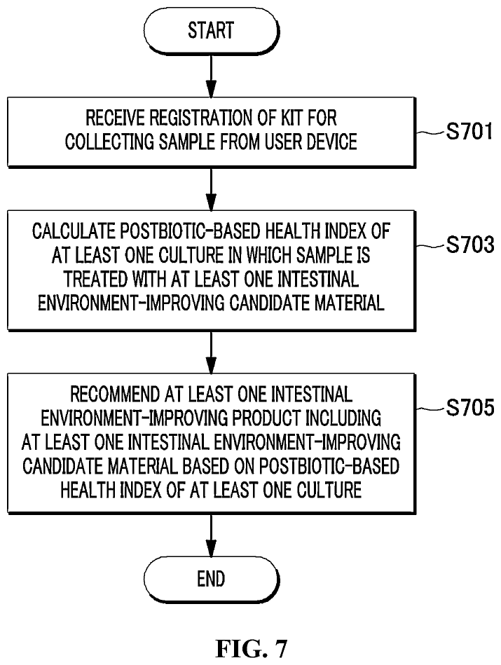

FIG. 7 is a flowchart showing a method for providing a personalized solution for improving an intestinal environment according to an embodiment of the present disclosure.

Referring to FIG. 7, in a process S701, the solution providing server 100 may receive registration of a kit for collecting a sample from the user device 110.

In a process S703, the solution providing server 100 may calculate a postbiotic-based health index of at least one culture in which a sample is treated with at least one intestinal environment-improving candidate material. Herein, the postbiotic-based health index may be calculated based on the ratio of short chain fatty acids in the culture. Also, the postbiotic-based health index may be calculated based on the ratio of acetic acid to propionic acid and butyric acid constituting the short chain fatty acids in the culture.

In a process S705, the solution providing server 100 may recommend at least one product for improving an intestinal environment, including at least one intestinal environment-improving candidate material based on the postbiotic-based health index of the at least one culture. Herein, the product for improving an intestinal environment may include at least one of probiotics, prebiotics, food, health functional food and drug.

In the descriptions above, the processes S701 through S705 may be divided into additional processes or combined into fewer processes depending on an exemplary embodiment. In addition, some of the processes may be omitted and the sequence of the processes may be changed if necessary.

An aspect of the present disclosure can be embodied in a storage medium including instruction codes executable by a computer such as a program module executed by the computer. A computer-readable medium can be any usable medium which can be accessed by the computer and includes all volatile/non-volatile and removable/non-removable media. Further, the computer-readable medium may include all computer storage media. The computer storage medium includes all volatile/non-volatile and removable/non-removable media embodied by a certain method or technology for storing information such as computer-readable instruction code, a data structure, a program module or other data.

The above description of the present disclosure is provided for the purpose of illustration, and it would be understood by a person with ordinary skill in the art that various changes and modifications may be made without changing technical conception and essential features of the present disclosure. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. A method for providing a personalized solution for improving an intestinal environment based on a postbiotic-based health index, comprising:
receiving registration of a kit for collecting a sample from a user device;
calculating a postbiotic-based health index of at least one culture in which the sample is treated with at least one intestinal environment-improving candidate material; and
recommending at least one product for improving an intestinal environment, including the at least one intestinal environment-improving candidate material based on the postbiotic-based health index of the at least one culture.

2. The method for providing a personalized solution for improving an intestinal environment of claim 1,
wherein the postbiotic-based health index is based on a ratio of short chain fatty acids in the culture.

3. The method for providing a personalized solution for improving an intestinal environment of claim 2,
wherein the postbiotic-based health index is based on a ratio of acetic acid (acetate) to propionic acid (propionate) and butyric acid (butyrate).

4. The method for providing a personalized solution for improving an intestinal environment of claim 1,
wherein the calculating a postbiotic-based health index includes:
selecting a culture having a greatest difference between a postbiotic-based health index of a control group in which the sample is not treated with any intestinal environment-improving candidate material and the postbiotic-based health index of the at least one culture, and
the recommending at least one product for improving an intestinal environment includes:
recommending a product for improving an intestinal environment, including the intestinal environment-improving candidate material used for culturing the culture having the greatest difference.

5. The method for providing a personalized solution for improving an intestinal environment of claim 1,
wherein the product for improving an intestinal environment includes at least one of probiotics, prebiotics, food, health functional food and drug.

6. A solution providing server for providing a personalized solution for improving an intestinal environment based on a postbiotic-based health index, comprising:
a kit registration unit that receives registration of a kit for collecting a sample from a user device;
a postbiotic-based health index calculation unit that calculates a postbiotic-based health index of at least one culture in which the sample is treated with at least one intestinal environment-improving candidate material; and
a product recommendation unit that recommends at least one product for improving an intestinal environment, including the at least one intestinal environment-improving candidate material based on the postbiotic-based health index of the at least one culture.

7. The solution providing server of claim 1,
wherein the postbiotic-based health index is based on a ratio of short chain fatty acids in the culture.

8. The solution providing server of claim 7,
wherein the postbiotic-based health index is based on a ratio of acetic acid (acetate) to propionic acid (propionate) and butyric acid (butyrate).

9. The solution providing server of claim 6,
wherein the postbiotic-based health index calculation unit selects a culture having a greatest difference between postbiotic-based health index of a control group in which the sample is not treated with any intestinal environment-improving candidate material and the postbiotic-based health index of the at least one culture, and
the product recommendation unit recommends a product for improving an intestinal environment, including the intestinal environment-improving candidate material used for culturing the culture having the greatest difference.

10. The solution providing server of claim 6,
wherein the product for improving an intestinal environment includes at least one of probiotics, prebiotics, food, health functional food and drug.
